(19) United States
(12) Patent Application Publication
(10) Pub. No.: US 2005/0215802 A1
(43) Pub. Date: Sep. 29, 2005

(54) Process for preparing an alkylene oxide

(76) Inventors: Gerard Johan Heiszwolf, Heiloo (NL); Enno Boelo De Vries, Vondelingenplaat (NL)

Correspondence Address: SHELL OIL COMPANY, P O BOX 2463, HOUSTON, TX, 772522463, US

(21) Appl. No.: 11/088,399

(22) Filed: Mar. 24, 2005

(30) Foreign Application Priority Data
Mar. 26, 2004 (EP) 04251780.5

(51) Int. Cl. C07D 301/19

(52) U.S. Cl. 549/529

(57) ABSTRACT

The invention relates to a process for preparing an alkylene oxide, which process comprises: (a) oxidizing of an organic compound to obtain reaction product containing organic hydroperoxide; (b) washing at least part of the organic hydroperoxide containing reaction product with a basic aqueous solution; (c) separating the mixture obtained in step (b) into a hydrocarbonaceous phase and an aqueous phase; (d) washing at least part of the hydrocarbonaceous phase obtained in step (c) with water; (e) separating the mixture obtained in step (d) into a hydrocarbonaceous phase and an aqueous phase; and, (f) contacting at least part of the hydrocarbonaceous phase obtained in step (e) with an alkene and catalyst to obtain an alkylene oxide, in which process the separation of hydrocarbonaceous phase and aqueous phase of step (c) and/or (e) is carried out with the help of a coalescer containing polypropylene fibers.

### REFERENCE TO EARLIER APPLICATION

This application claims priority under 35 U.S.C. §119 to EP 04251780.5 filed on Mar. 26, 2004.

### FIELD OF THE INVENTION

The present invention relates to a process for preparing an alkylene oxide employing an organic hydroperoxide.

### BACKGROUND OF THE INVENTION

Processes for preparing alkylene oxide, and especially propylene oxide, employing organic hydroperoxides, are well known in the art. As described in U.S. Pat. No. 5,883,268, a process for preparing propylene oxide can comprise peroxidation of ethylbenzene, followed by contacting the peroxidation reaction product with aqueous base in an amount sufficient to neutralize acidic components thereof and separating the resulting mixture into an aqueous stream and a deacidified organic stream. The base contaminated, deacidified hydroperoxide stream is washed with water. A similar process is described in WO-A-03/066584. In such processes, the organic phase has to be separated from aqueous phase. The separation can be carried out efficiently with the help of coalescers.

Coalescers comprise fibers which promote the growth of droplets in a dispersion. However, conventional coalescers tend to lose their mechanical strength if used for separating the organic phase from the aqueous phase in the process of the present invention. Further, unacceptable decomposition of the organic hydroperoxide has been observed in some instances.

Polypropylene fibers are generally not used in processes in which they would be in contact with aromatic compounds as the fibers tend to swell in such environments to such degree that their mechanical properties become unacceptable.

### SUMMARY OF THE INVENTION

The present invention is directed to a process for preparing an alkylene oxide, which process comprises:
(a) oxidizing an organic compound to obtain a reaction product containing organic hydroperoxide,
(b) washing at least part of the organic hydroperoxide containing reaction product with a basic aqueous solution,
(c) separating the mixture obtained in step (b) into a hydrocarbonaceous phase and an aqueous phase,
(d) washing at least part of the hydrocarbonaceous phase obtained in step (c) with water,
(e) separating the mixture obtained in step (d) into a hydrocarbonaceous phase and an aqueous phase, and
(f) contacting at least part of the hydrocarbonaceous phase obtained in step (e) with an alkene and catalyst to obtain an alkylene oxide,
   in which process the separation of the hydrocarbonaceous phase and the aqueous phase of step (c) and/or (e) is performed with a coalescer containing polypropylene fibers.

### DETAILED DESCRIPTION OF THE INVENTION

It has now been found that coalescers containing polypropylene fibers are suitable for separating the aqueous phase from the hydrocarbonaceous phase in the process of the present invention while maintaining their mechanical strength. Additionally, polypropylene fibers were not found to increase decomposition of the hydroperoxide or only to a very limited extent. It was also found that the polypropylene fibers do not decompose in solutions of ethylbenzenehydroperoxide in ethylbenzene under the reaction conditions applied in the present process.

The alkene used in the process according to the invention is preferably an alkene comprising from 2 to 10 carbon atoms and more preferably an alkene comprising from 2 to 4 carbon atoms. The corresponding prepared alkylene oxide preferably also comprises from 2 to 10 carbon atoms and more preferably from 2 to 4 carbon atoms. Examples of alkenes that may be used include ethene, propene, 1-butene and 2-butene, with which the corresponding ethylene oxide, propylene oxide and butylene oxides may be prepared.

The process according to the invention is especially useful for the preparation of propylene oxide. Hence, the most preferred alkene is propene, with which the corresponding propylene oxide may be prepared.

Although the organic compound used in the process of the present invention may in principle be any organic compound, organic compounds which are preferred are alkylaryl compounds. Alkylaryl compounds which are preferred are benzene compounds containing at least 1 alkyl substituent which alkyl substituent contains from 1 to 10 carbon atoms, preferably from 2 to 8 carbon atoms. Preferably, the benzene compound contains on average from 1 to 2 constituents. Preferred benzene compounds are ethylbenzene, cumene and di(iso-propyl)benzene.

The oxidation of the organic compound may be carried out by any suitable process known in the art. The oxidation may be carried out in the liquid phase in the presence of a diluent. This diluent is preferably a compound which is liquid under the reaction conditions and does not react with the starting materials and product obtained. However, the diluent may also be a compound necessarily present during the reaction. For example, if the alkylaryl is ethylbenzene the diluent may be ethylbenzene as well.

Besides the desired organic hydroperoxide, a wide range of contaminants may be created during the oxidation of organic compounds. Although most of these are present in small amounts, the presence of organic acids, in particular, has been found to sometimes cause problems in the subsequent use of the organic hydroperoxides. As described in U.S. Pat. No. 5,883,268, a method of reducing the amount of contaminants is contacting the reaction product containing organic hydroperoxide with an aqueous alkali solution. However, contact with the aqueous alkali solution introduces a certain amount of alkali metal into the organic hydroperoxide containing reaction product.

In the process of the present invention, the organic hydroperoxide containing reaction product is contacted with a basic aqueous solution, more specifically a basic aqueous solution containing one or more alkali metal compounds. Suitable alkali sources for use in the aqueous alkali solution include alkali metal hydroxides, alkali metal carbonates and alkali metal hydrogen carbonates. Examples of these compounds are NaOH, KOH, Na₂CO₃, K₂CO₃, NaHCO₃ and KHCO₃. In view of their easy availability, it is preferred to use NaOH and/or Na₂CO₃.

In steps (c) and (e), the hydrocarbonaceous phase containing the organic hydroperoxide is separated from the aqueous phase. A preferred method comprises allowing the hydrocarbonaceous phase and aqueous phase to settle in a settling vessel and subsequently separating a hydrocarbonaceous phase from an aqueous phase. In such case, step (c) and/or (e) would comprise:
(1) allowing the mixture obtained to settle in a settler,
(2) removing the hydrocarbonaceous phase and the aqueous phase from the settler, and
(3) treating the hydrocarbonaceous phase obtained in step (2) in a coalescer containing polypropylene fibers to obtain a dry hydrocarbonaceous phase.

It is preferred that at least step (c) is carried out within a coalescer containing polypropylene fibers as it is thought that the decrease in mechanical strength of the coalescer fibers is caused by the contact between fibers and basic aqueous solution.

The polypropylene fiber to be applied in the present invention may in principle be any fiber. However, it is preferred that the polypropylene fiber is free of phosphorus and/or sulfur containing additives. It was found that in some cases, these additives could lead to increased decomposition of the organic hydroperoxide. Polypropylene fibers which were found to be suitable are fibers made from polypropylene containing less than 1000 ppm of sulfur, based on amount of elemental sulfur on total amount of polypropylene. The amount of phosphorus, based on amount of elemental phosphorus on total amount of polypropylene, is preferably at most 1000 ppm. Most preferably the amount of sulfur is at most 290 ppm while additionally the amount of phosphorus is at most 250 ppm. The polypropylene preferably is an isotactic homopolymer.

It is preferred to use carded polypropylene fibers in the process of the present invention. Carding of fibers comprises separating and opening fiber bundles into individual fibers and provides drafting, orientation and/or randomization of the individual fibers.

Preferably, the separation of hydrocarbonaceous phase and aqueous phase of step (c) and/or (e) is carried out at a temperature of between 0° C. and 80° C.

The coalescer for use in the present invention may be any coalescer known to be suitable to someone skilled in the art. Coalescers which may be used are vertical or horizontal vessels containing a bed or mat comprising or consisting of polypropylene fibers. In such vessels, the mixture of hydrocarbonaceous and/or aqueous phase is passed through the bed or mat. Another type of coalescers are coalescers containing internals comprising or consisting of polypropylene fibers through which the mixture of hydrocarbonaceous and/or aqueous phase is passed. Such internals are sometimes called cartridges. The presence of internals may be advantageous if a larger contact area is desired. A larger contact area allows lower space velocities.

It may be advantageous to filter the mixture of hydrocarbonaceous and aqueous phase before contact with coalescers containing internals. Such filters generally have openings of at most 20 micrometers, preferably of at most 10 micrometers.

The coalescer for use in the present invention may be used in the conventional way as is known to those skilled in the art. It is customary to monitor the pressure drop over the bed or mat of fibers during operation. If the pressure drop has become unacceptable, the bed or mat may be cleaned for example by back-washing.

In step (d), at least part of the separated hydro-carbonaceous phase obtained is washed with water. The water may be clean water but preferably consists at least partly of waste water. The washing will generally be carried out with a combination of fresh water, recycle water and optionally further waste water obtained in other steps of the present process.

After step (d), hydrocarbonaceous phase is separated from aqueous phase in step (e).

Dependent on the amount of contaminants present in the hydrocarbonaceous phase containing organic peroxide, process step (d) and (e) may either be carried out once or a number of times. Preferably, the combination of these process steps is carried out from 1 to 3 times.

In process step (f), at least part of the hydrocarbonaceous phase containing organic hydroperoxide obtained in step (e) is contacted with an alkene, in the presence of a catalyst to obtain an alkylene oxide. The organic hydroperoxide is converted into an alcohol. A catalyst which may suitably be used in such process comprises titanium on silica and/or silicate. A preferred catalyst is described in EP-A-345856. The reaction generally proceeds at moderate temperatures and pressures, in particular at temperatures in the range of from 0° C. to 200° C., preferably in the range from 25° C. to 200° C. The precise pressure is not critical as long as it suffices to maintain the reaction mixture as a liquid or as a mixture of vapor and liquid. Atmospheric pressure may be satisfactory. In general, pressures can be in the range of from 1 to 100×10⁵ N/m².

The alkylene oxide can be separated from the reaction product in any way known to be suitable to someone skilled in the art. The liquid reaction product may be worked up by fractional distillation, selective extraction and/or filtration. The solvent, the catalyst and any unreacted alkene or hydroperoxide may be recycled for further utilization.

Preferably, the organic compound for use in the present invention is ethylbenzene and such process generally further comprises:
(g) separating at least part of the alkylene oxide from the reaction mixture comprising 1-phenyl-ethanol, and
(h) converting at least part of the 1-phenylethanol into styrene.

Processes which may be used for this step have been described in WO 99/42425 and WO 99/42426. However, any suitable process known to someone skilled in the art can in principle be used.

The present invention is further illustrated by the following examples.

### EXAMPLE 1

In a reactor, air was blown through ethylbenzene. The product obtained was distilled such as to obtain a mixture containing about 25% wt of ethylbenzene hydroperoxide (EBHP) in ethylbenzene. Additionally, by-products will be present in this mixture.

A basic aqueous solution was prepared by mixing 65 grams of Na₂CO₃, 1000 grams of water and 65 grams benzoic acid. This solution had a pH of 8.5-9.0.

The polymer fibers were contacted at 80° C. for 1 month with a mixture of 600 ml of the ethylbenzene hydroperoxide solution and 300 ml of the Na₂CO₃ solution. After 1 month, the tenacity at break of the polymer fibers was as described in Table 1.

Additionally, the tenacity at break of the polymer is included as obtained from the supplier.

TABLE 1

| | Tenacity at break | Tenacity at break |
|---|---|---|
| | before contact | after contact |
| Polymer | (g/dtex) | (g/dtex) |
| Polypropylene | 2.16 | 2.13 |
| Polyester^{a} | 3.29 | 0.35 |
| Polyamide^{b} | 1.81 | 1.47 |
| Polyacrylonitrile | 2.83 | 1.26 |
| Polyamide^{c} | * | * |
| Cellulose | | ** |

*: Not measured. No substantial decrease observed
**: Decomposed into a pulp-like mass
^{a}Trevira type 813 ex Hoechst
^{b}a polyamide made from diaminobenzene and terephtalic acid
^{c}nylon-6

### EXAMPLE 2

The influence of the polymer fiber on the EBHP solution was measured by bringing the fiber into contact with 20% wt of ethylbenzene hydroperoxide in ethylbenzene at 80° C.

Decomposition in the presence of polyester fiber and cellulose was not measured as the mechanical strength of these fibers was unacceptable.

The data in Table 2 are the amount of decomposition products compounds present in the solution after 235 hours, with the exception of the testing of nylon-6 which was shorter (72 hours).

TABLE 2

| | amount of compounds present in solution (% wt) | | | |
|---|---|---|---|---|
| | methylphenyl- | 1- | benz- | |
| Polymer | ketone | phenylethanol | aldehyde | phenol |
| Polypropylene | 1.89 | 2.78 | 0.06 | 0.14 |
| Polyamide^{b} | 1.99 | 2.70 | 0.07 | 0.21 |
| Polyacrylonitrile | 2.07 | 3.15 | 0.06 | 0.18 |
| Polyamide^{c} | 4.20*** | 5.0*** | 0.07 | 0.08 |

^{b}a polyamide made from diaminobenzene and terephtalic acid
^{c}nylon-6
***measured after 72 hours

1. A process for preparing an alkylene oxide, which process comprises:
(a) oxidizing an organic compound to obtain reaction product containing organic hydroperoxide;
(b) washing at least part of the organic hydroperoxide containing reaction product with a basic aqueous solution;
(c) separating the mixture obtained in step (b) into a hydrocarbonaceous phase and an aqueous phase;
(d) washing at least part of the hydrocarbonaceous phase obtained in step (c) with water;
(e) separating the mixture obtained in step (d) into a hydrocarbonaceous phase and an aqueous phase; and,
(f) contacting at least part of the hydrocarbonaceous phase obtained in step (e) with an alkene and catalyst to obtain an alkylene oxide, in which process the separation of hydrocarbonaceous phase and aqueous phase of step (c) and/or (e) is carried out with a coalescer containing polypropylene fibers.

2. The process of claim 1, in which process the separation of step (c) is carried out with a coalescer containing polypropylene fibers.

3. The process of claim 2, in which process the separation of hydrocarbonaceous phase and aqueous phase is carried out by
(1) allowing the mixture obtained to settle in a settler;
(2) removing the hydrocarbonaceous phase and the aqueous phase from the settler; and,
(3) treating the hydrocarbonaceous phase obtained in step (2) in a coalescer containing polypropylene fibers to obtain a dry hydrocarbonaceous phase.

4. The process of claim 2, in which process the water used for washing consists at least partly of waste water.

5. The process for preparing an alkylene oxide of claim 2, in which process the organic compound is ethylbenzene and which process further comprises:
(g) separating at least part of the alkylene oxide from the reaction mixture comprising 1-phenylethanol; and,
(h) converting at least part of the 1-phenylethanol into styrene.

6. The process of claim 2, in which process the alkene is propene and the alkylene oxide is propylene oxide.

7. The process of claim 1, in which process the separation of hydrocarbonaceous phase and aqueous phase is carried out by
(1) allowing the mixture obtained to settle in a settler;
(2) removing the hydrocarbonaceous phase and the aqueous phase from the settler; and,
(3) treating the hydrocarbonaceous phase obtained in step (2) in a coalescer containing polypropylene fibers to obtain a dry hydrocarbonaceous phase.

8. The process of claim 7, in which process the water used for washing consists at least partly of waste water.

9. The process of claim 7, in which process the alkene is propene and the alkylene oxide is propylene oxide.

10. The process of claim 1, in which process the water used for washing consists at least partly of waste water.

11. The process of claim 10, in which process the alkene is propene and the alkylene oxide is propylene oxide.

12. The process for preparing an alkylene oxide of claim 1, in which process the organic compound is ethylbenzene and which process further comprises:
(g) separating at least part of the alkylene oxide from the reaction mixture comprising 1-phenylethanol; and,
(h) converting at least part of the 1-phenylethanol into styrene.

13. The process of claim 12, in which process the alkene is propene and the alkylene oxide is propylene oxide.

14. The process of claim 1, in which process the alkene is propene and the alkylene oxide is propylene oxide.
